(19) United States
(12) Patent Application Publication
(10) Pub. No.: US 2024/0277259 A1
(43) Pub. Date: Aug. 22, 2024

(54) GAIT INDEX CALCULATION DEVICE, GAIT MEASUREMENT SYSTEM, GAIT INDEX CALCULATION METHOD, AND RECORDING MEDIUM

(71) Applicant: NEC Corporation, Tokyo (JP)

(72) Inventors: Fumiyuki NIHEY, Tokyo (JP); Chenhui Huang, Tokyo (JP); Kazuki Ihara, Tokyo (JP); Hiroshi Kajitani, Tokyo (JP)

(73) Assignee: NEC Corporation, Tokyo (JP)

(21) Appl. No.: 18/410,586

(22) Filed: Jan. 11, 2024

(30) Foreign Application Priority Data
Feb. 17, 2023 (JP) 2023-023187

(51) Int. Cl. A61B 5/11; A61B 5/00; A61B 5/103

(52) U.S. Cl. CPC A61B 5/112; A61B 5/1038; A61B 5/6807; A61B 5/7267

(57) ABSTRACT

Provided is a gait index calculation device that includes a communication unit that receives sensor data based on spatial acceleration and spatial angular velocity that are measured at a frequency lower than a normal measurement frequency, an interpolation unit that interpolates the sensor data using a predetermined interpolation method, a gait index calculation unit that calculates a gait index using the interpolated sensor data, and an output unit that outputs the calculated gait index.

This application is based upon and claims the benefit of priority from Japanese Patent Application No. 2023-023187, filed on Feb. 17, 2022, the disclosure of which is incorporated herein in its entirety by reference.

### TECHNICAL FIELD

The present disclosure relates to a gait index calculation device, a gait measurement system, a gait index calculation method, and a recording medium.

### BACKGROUND ART

With growing interest in healthcare, services that provide information regarding a gait have attracted attention. For example, a technique for analyzing a gait using sensor data measured by a sensor mounted on footwear such as shoes has been developed. In the time-series data of the sensor data, features regarding the gait appear. By using such features, an index regarding the gait (also referred to as a gait index) can be calculated.

PTL 1 (JP 2021-121807 A) discloses a technique intended to extend a battery life so that a wearable device can be used a longer period of time. The wearable device of PTL 1 includes a position detection unit, a sensor unit including at least one of an acceleration sensor, a geomagnetic sensor, and an atmospheric pressure sensor, and a processing unit electrically connected to the position detection unit and the sensor unit. The processing unit determines a behavior state of the user based on signals from the position detection unit and the sensor unit. The processing unit obtains position information of the user by controlling at least two of the position detection unit, the acceleration sensor, the geomagnetic sensor, and the atmospheric pressure sensor based on the behavior state.

PTL 2 (WO 2021/210172 A1) discloses a data processing device intended to interpolate data loss regarding a living body. PTL 2 discloses transmitting, to a terminal device, sensor data generated by using a sensor value acquired by a sensor included in a gait measurement device provided to an insole.

The wearable device of PTL 1 reduces power consumption by controlling operations of the position detection unit and the various sensors using the acceleration sensor, geomagnetic sensor, and atmospheric pressure sensor having relatively low power consumption. As a result, according to the method of PTL 1, the battery life is longer than that of the general method, and the wearable device can be used for a longer time. In the method of PTL 1, it is needed to detect a position by a global positioning system (GPS), wireless communication radio waves, wireless positioning that calculates an absolute position in cooperation with a network, or the like. In other words, in the method of PTL 1, power consumed for GPS and wireless positioning is required.

In the method of PTL 2, the power of the gait measurement device provided to the insole is consumed in order to transmit the sensor data used for calculating the gait index to the terminal device. In order to use the gait measurement device provided to the insole for a longer time, it is desired to suppress the power consumption as much as possible. For example, if the gait measurement device provided to the insole calculates the gait index (including the intermediate result) and transmits the calculated gait index to the smart phone, the communication amount and the power consumption can be reduced as compared with the case of transmitting the sensor data as it is to the terminal device. However, when the number of gait indexes increases, a calculation load of the gait measurement device provided to the insole increases, and a communication amount and power consumption increase.

An object of the present disclosure is to provide a gait index calculation device and the like capable of reducing a communication load of sensor data and calculating a gait index with sufficient accuracy.

### SUMMARY

A gait index calculation device according to an aspect of the present disclosure includes a communication unit that receives sensor data based on spatial acceleration and spatial angular velocity that are measured at a frequency lower than a normal measurement frequency, an interpolation unit that interpolates the sensor data using a predetermined interpolation method, a gait index calculation unit that calculates a gait index using the interpolated sensor data, and an output unit that outputs the calculated gait index.

A gait index calculation method according to an aspect of the present disclosure includes receiving sensor data based on spatial acceleration and spatial angular velocity that are measured at a frequency lower than a normal measurement frequency, interpolating the sensor data using a predetermined interpolation method, calculating a gait index using the interpolated sensor data, and outputting the calculated gait index.

A program according to one aspect of the present disclosure causes a computer to execute processing including receiving sensor data based on spatial acceleration and spatial angular velocity that are measured at a frequency lower than a normal measurement frequency, interpolating the sensor data using a predetermined interpolation method, calculating a gait index using the interpolated sensor data, and outputting the calculated gait index.

### BRIEF DESCRIPTION OF THE DRAWINGS

Exemplary features and advantages of the present invention will become apparent from the following detailed description when taken with the accompanying drawings in which:

FIG. 1 is a block diagram illustrating an example of a configuration of a gait measurement system according to the present disclosure;

FIG. 2 is a block diagram illustrating an example of a configuration of a measurement device included in the gait measurement system according to the present disclosure;

FIG. 3 is a conceptual diagram illustrating an arrangement example of measurement devices included in the gait measurement system according to the present disclosure;

FIG. 4 is a conceptual diagram for describing a coordinate system set in the measurement device included in the gait measurement system according to the present disclosure;

FIG. 5 is a conceptual diagram for describing human body planes regarding the gait measurement system according to the present disclosure;

FIG. 6 is a conceptual diagram for describing a gait cycle regarding the gait measurement system according to the present disclosure;

FIG. 7 is a block diagram illustrating an example of a configuration of a gait index calculation device included in the gait measurement system according to the present disclosure;

FIG. 8 is a graph illustrating an example of acceleration in the left-right direction measured by the measurement device included in the gait measurement system according to the present disclosure;

FIG. 9 is a graph illustrating an example of traveling direction acceleration measured by the measurement device included in the gait measurement system according to the present disclosure;

FIG. 10 is a graph illustrating an example of vertical direction acceleration measured by the measurement device included in the gait measurement system according to the present disclosure;

FIG. 11 is a graph illustrating an example of angular velocity about the left-right direction axis measured by the measurement device included in the gait measurement system according to the present disclosure;

FIG. 12 is a graph illustrating an example of angular velocity about the traveling direction axis measured by the measurement device included in the gait measurement system according to the present disclosure;

FIG. 13 is a graph illustrating an example of angular velocity about the vertical direction axis measured by the measurement device included in the gait measurement system according to the present disclosure;

FIG. 14 is a graph for describing linear interpolation by the gait index calculation device included in the gait measurement system according to the present disclosure;

FIG. 15 is a graph for describing polynomial interpolation by the gait index calculation device included in the gait measurement system according to the present disclosure;

FIG. 16 is a conceptual diagram for describing machine learning interpolation by the gait index calculation device included in the gait measurement system according to the present disclosure;

FIG. 17 is a graph for describing the machine learning interpolation by the gait index calculation device included in the gait measurement system according to the present disclosure;

FIG. 18 is a conceptual diagram for describing an example of a gait index calculated by the gait index calculation device included in the gait measurement system according to the present disclosure;

FIG. 19 is a flowchart for describing an example of the operation of the measurement device included in the gait measurement system according to the present disclosure;

FIG. 20 is a flowchart for describing an example of the operation of the gait index calculation device included in the gait measurement system according to the present disclosure;

FIG. 21 is a conceptual diagram for describing an application example of the gait measurement system according to the present disclosure;

FIG. 22 is a block diagram illustrating an example of a configuration of the gait index calculation device included in the gait measurement system according to the present disclosure; and

FIG. 23 is a block diagram illustrating an example of a hardware configuration for executing control and processing according to the present disclosure.

### EXAMPLE EMBODIMENT

Example embodiments of the present invention will be described below with reference to the drawings. In the following example embodiments, technically preferable limitations are imposed to carry out the present invention, but the scope of this invention is not limited to the following description. In all drawings used to describe the following example embodiments, the same reference numerals denote similar parts unless otherwise specified. In addition, in the following example embodiments, a repetitive description of similar configurations or arrangements and operations may be omitted. Further, the directions of the arrows in the drawings are examples and are not illustrated to limit the directions of the signals between the blocks.

### First Example Embodiment

First, a gait measurement system according to a first example embodiment will be described with reference to the drawings. The gait measurement system according to the present example embodiment measures sensor data associated to a gait of a user. The gait measurement system according to the present example embodiment calculates an index (gait index) regarding a feature (gait) included in the gait pattern of the user by using the measured sensor data.

### Configuration

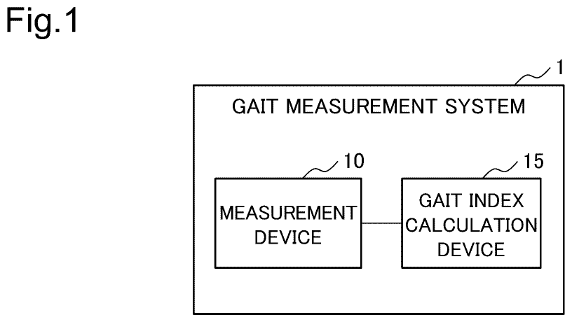

FIG. 1 is a block diagram illustrating an example of a configuration of a gait measurement system 1 according to the present disclosure. The gait measurement system 1 includes a measurement device 10 and a gait index calculation device 15. In the present example embodiment, an example in which the measurement device 10 and the gait index calculation device 15 are configured as separate hardware will be described. For example, the measurement device 10 is provided to footwear or the like of a subject (user) whose gait index is to be calculated. For example, the function of the gait index calculation device 15 is installed in a mobile terminal carried by the subject. The function of the gait index calculation device 15 may be implemented in a cloud or a server connected, via a network, to the mobile terminal carried by the subject.

Hereinafter, the configurations of the measurement device 10 and the gait index calculation device 15 will be individually described.

### [Measurement Device]

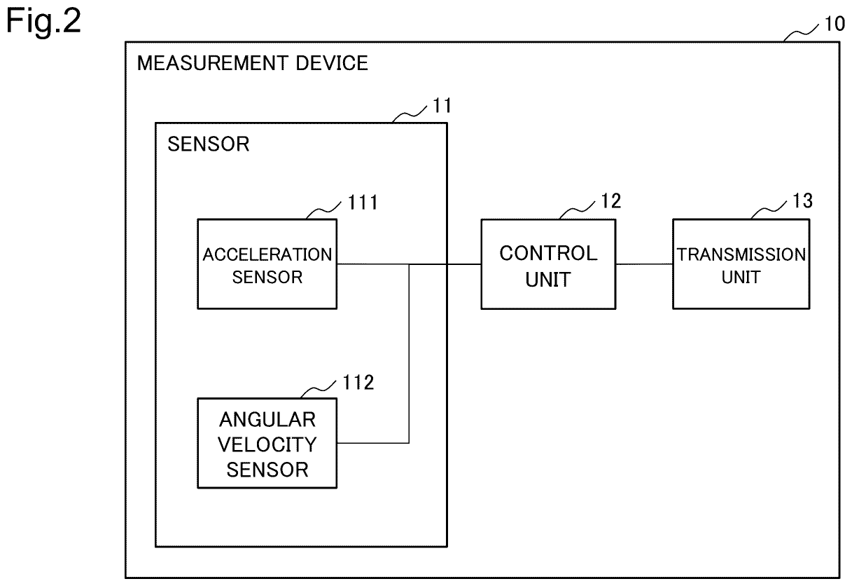

Next, a detailed configuration of the measurement device 10 will be described with reference to the drawings. FIG. 2 is a block diagram illustrating an example of a configuration of the measurement device 10. The measurement device 10 includes a sensor 11, a control unit 12, and a transmission unit 13. As illustrated in FIG. 2, the sensor 11 includes an acceleration sensor 111 and an angular velocity sensor 112. FIG. 2 illustrates an example in which the acceleration sensor 111 and the angular velocity sensor 112 are included in the sensor 11. The sensor 11 may include a sensor other than the acceleration sensor 111 and the angular velocity sensor 112. Sensors other than the acceleration sensor 111 and the angular velocity sensor 112 that can be included in the sensor 11 will not be described.

The sensor 11 measures acceleration and angular velocity at a frequency lower than a normal measurement frequency. Normally, in order to accurately calculate the gait index, the measurement frequency may be about 100 Hz (hertz). For example, the sensor 11 measures acceleration and angular velocity at a low frequency of about 25 Hz. When the measurement frequency is about 25 Hz, the gait index cannot be accurately calculated. Therefore, in order to accurately calculate the gait index, the sensor data based on the acceleration and the angular velocity measured by the sensor 11 is interpolated by the gait index calculation device 15.

The acceleration sensor 111 is a sensor that measures acceleration (also referred to as spatial acceleration) in three axial directions. The acceleration sensor 111 measures acceleration (also referred to as spatial acceleration) as a physical quantity related to the movement of a foot. The acceleration sensor 111 measures acceleration at a frequency lower than a normal measurement frequency. For example, the acceleration sensor 111 measures acceleration at 25 Hz. The acceleration sensor 111 outputs the measured acceleration to the transmission unit 13. For example, a sensor of a piezoelectric type, a piezoresistive type, a capacitance type, or the like can be used as the acceleration sensor 111. The sensor used as the acceleration sensor 111 is not limited to the measurement method as long as the sensor can measure acceleration.

The angular velocity sensor 112 is a sensor that measures angular velocity (also referred to as spatial angular velocity) about three axes. The angular velocity sensor 112 measures angular velocity (also referred to as spatial angular velocity) as a physical quantity related to the movement of a foot. The angular velocity sensor 112 measures an angular velocity at a frequency lower than a normal measurement frequency. For example, the angular velocity sensor 112 measures the angular velocity at 25 Hz. The angular velocity sensor 112 outputs the measured angular velocity to the transmission unit 13. For example, a sensor of a vibration type, a capacitance type, or the like can be used as the angular velocity sensor 112. The sensor used as the angular velocity sensor 112 is not limited to the measurement method as long as the sensor can measure the angular velocity.

The sensor 11 is achieved by, for example, an inertial measurement device that measures acceleration and angular velocity. An example of the inertial measurement device is an inertial measurement unit (IMU). The IMU includes an acceleration sensor 111 that measures acceleration in three axis directions and the angular velocity sensor 112 that measures angular velocities about the three axes. The sensor 11 may be achieved by an inertial measurement device such as a vertical gyro (VG) or an attitude heading reference system (AHRS). The sensor 11 may be achieved by a global positioning system/inertial navigation system (GPS/INS). The sensor 11 may be achieved by a device other than the inertial measurement device as long as it can measure a physical quantity related to the movement of the foot.

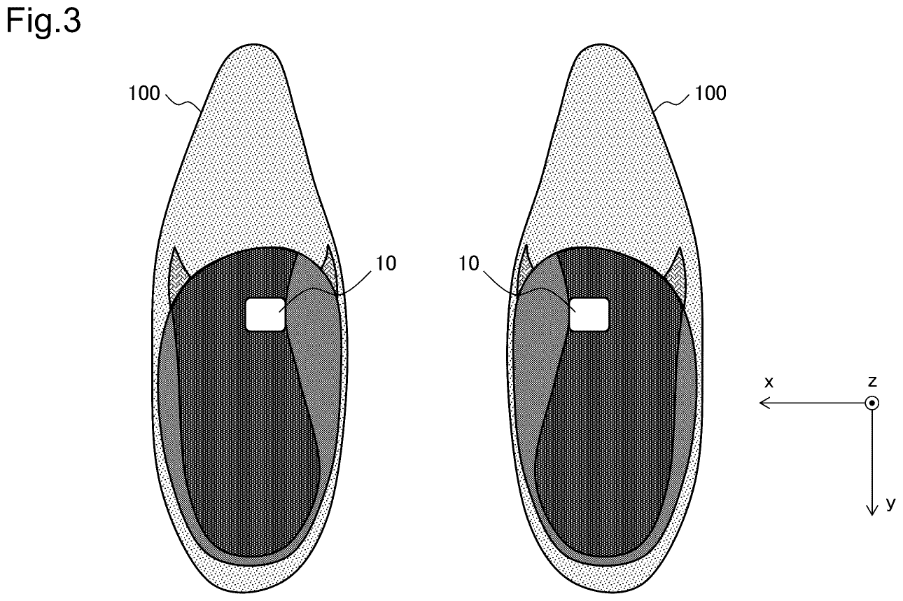

FIG. 3 is a conceptual diagram illustrating an example in which the measurement devices 10 are provided in the shoes 100 of both feet. In the example of FIG. 3, the measurement device 10 is installed on a position relevant to the back side of the arch of foot. For example, the measurement device 10 is installed on an insole inserted into the shoe 100. For example, the measurement device 10 may be provided to the bottom surface of the shoe 100. For example, the measurement device 10 may be embedded in the main body of the shoe 100. The measurement device 10 may be detachable from the shoe 100 or may not be detachable from the shoe 100. The measurement device 10 may be installed at a position other than the back side of the arch of the foot as long as the sensor data regarding the movement of the foot can be measured. The measurement device 10 may be provided to a sock worn by the user or a decorative article such as an anklet worn by the user. In addition, the measurement device 10 may be directly attached to the foot or may be embedded in the foot. FIG. 3 illustrates an example in which the measurement devices 10 are provided to the shoes 100 of both feet. The measurement device 10 may be provided to the shoe 100 of one foot.

In the example of FIG. 3, a local coordinate system including an x-axis in the horizontal direction, a y-axis in the front-rear direction, and a z-axis in the vertical direction is set with reference to the measurement device 10 (sensor 11). In the x-axis, the left side is positive, in the y-axis, the rear side is positive, and in the z-axis, the upper side is positive. The direction of the axes set in the sensor 11 may be the same for the left and right feet, or may be different for the left and right feet. For example, in a case where the sensors 11 produced with the same specifications are arranged in the left and right shoes 100, the vertical directions (directions in the Z-axis direction) of the sensors 11 arranged in the left and right shoes 100 are the same. In this case, the three axes of the local coordinate system set in the sensor data derived from the left foot and the three axes of the local coordinate system set in the sensor data derived from the right foot are the same on the left and right.

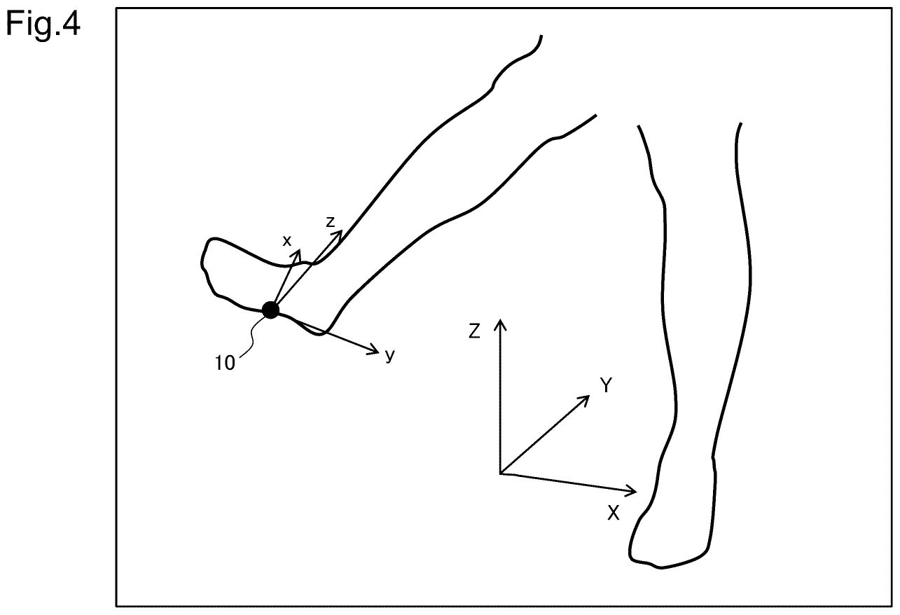

FIG. 4 is a conceptual diagram for describing a local coordinate system (x-axis, y-axis, z-axis) set in the measurement device 10 (sensor 11) installed on the back side of the arch of foot and a world coordinate system (X-axis, Y-axis, Z-axis) set with respect to the ground. In the world coordinate system (X-axis, Y-axis, Z-axis), in a state where the user facing the traveling direction is upright, the lateral direction of the user is set to the X-axis direction (the leftward direction is positive), the direction of the back of the user is set to the Y-axis direction (the rearward direction is positive), and the vertical direction is set to the Z-axis direction (the vertically upward direction is positive). The example of FIG. 4 conceptually illustrates the relationship between the local coordinate system (x-axis, y-axis, z-axis) and the world coordinate system (X-axis, Y-axis, Z-axis), and does not accurately illustrate the relationship between the local coordinate system and the world coordinate system that varies depending on the walking of the user.

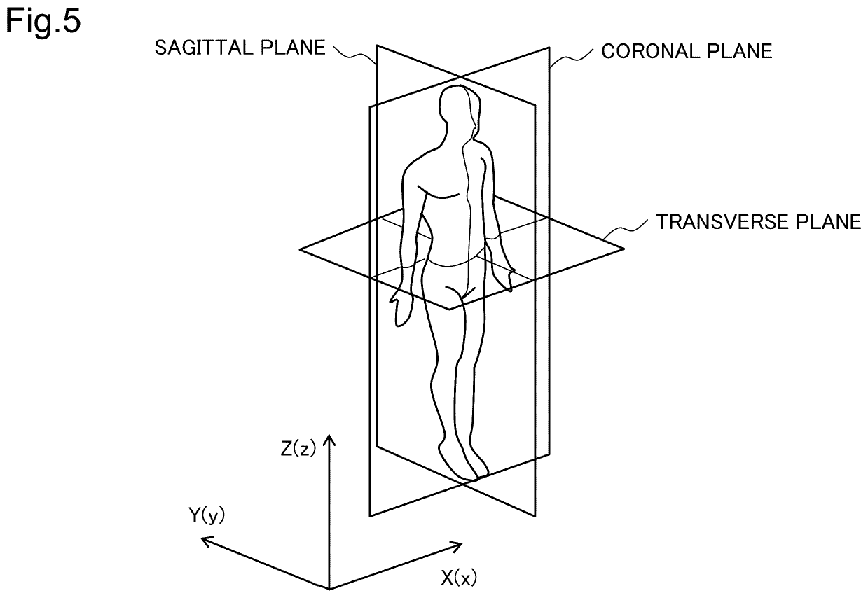

FIG. 5 is a conceptual diagram for describing planes (also referred to as human body planes) set for the human body. In the present example embodiment, a sagittal plane dividing the body into left and right, a coronal plane dividing the body into front and rear, and a transverse plane dividing the body horizontally are defined. As illustrated in FIG. 5, the world coordinate system and the local coordinate system coincide with each other in a state in which the center line of the foot is oriented in the traveling direction. In the present example embodiment, rotation in the sagittal plane with the x-axis as the rotation axis is defined as roll, rotation in the coronal plane with the y-axis as the rotation axis is defined as pitch, and rotation in the transverse plane with the z-axis as the rotation axis is defined as yaw. In addition, a rotation angle in a sagittal plane with the x-axis as a rotation axis is defined as a roll angle, a rotation angle in a coronal plane with the y-axis as a rotation axis is defined as a pitch angle, and a rotation angle in a transverse plane with the z-axis as a rotation axis is defined as a yaw angle.

The control unit 12 (control means) acquires acceleration in three axial directions from the acceleration sensor 111. The control unit 12 acquires the angular velocity about three axes from angular velocity sensor 112. For example, the control unit 12 performs analog-to-digital conversion (AD conversion) on the acquired physical quantities (analog data) such as angular velocity and acceleration. The physical quantities (analog data) measured by the acceleration sensor 111 and the angular velocity sensor 112 may be converted into digital data in each of the acceleration sensor 111 and the angular velocity sensor 112. The control unit 12 outputs the converted digital data (also referred to as sensor data) to the transmission unit 13.

The control unit 12 may be configured to store the sensor data in a storage unit (not illustrated). The sensor data includes at least acceleration data converted into digital data and angular velocity data converted into digital data. The acceleration data includes acceleration vectors in three axial directions. The angular velocity data includes angular velocity vectors about three axes. The acceleration data and the angular velocity data are associated with acquisition times of the data. The control unit 12 may add corrections such as a mounting error, temperature correction, and linearity correction to the acceleration data and the angular velocity data.

For example, the control unit 12 is achieved by a microcomputer or a microcontroller that performs overall control and data processing of the measurement device 10. For example, the control unit 12 includes a central processing unit (CPU), a random access memory (RAM), a read only memory (ROM), a flash memory, and the like. The control unit 12 controls the acceleration sensor 111 and the angular velocity sensor 112 to measure the angular velocity and the acceleration.

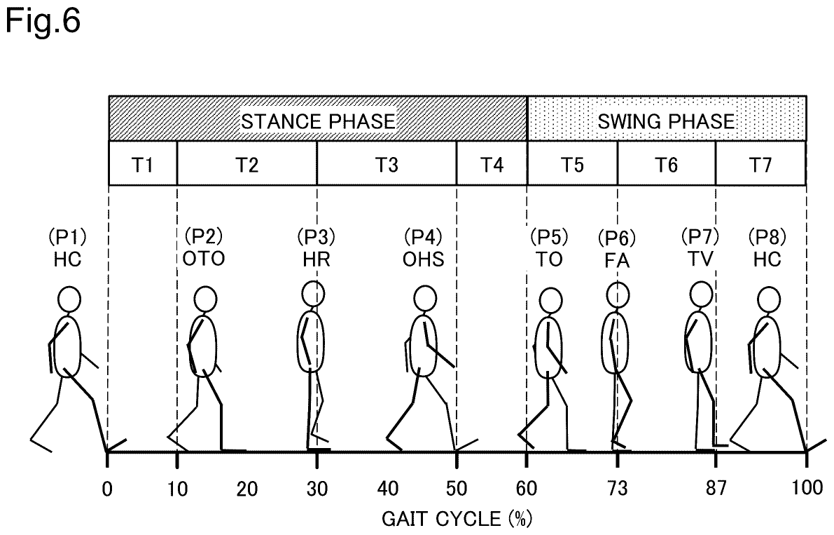

FIG. 6 is a conceptual diagram for explaining one gait cycle with the right foot as a reference. One gait cycle based on the left foot is also similar to that of the right foot. The left-right direction axis of FIG. 6 is one gait cycle of the right foot with a time point at which the heel of the right foot lands on the ground as a starting point and a time point at which the heel of the right foot next lands on the ground as an ending point. The left-right direction axis in FIG. 6 is first normalized with one gait cycle as 100%. In addition, in the left-right direction axis of FIG. 6, the second normalization is performed such that the stance phase is 60% and the swing phase is 40%. The one gait cycle of one foot is roughly divided into the stance phase in which at least a part of the back side of the foot is in contact with the ground and the swing phase in which the back side of the foot is not in contact with the ground. The stance phase is further subdivided into an initial stance period T1, a mid-stance period T2, a terminal stance period T3, and a pre-swing period T4. The swing phase is further subdivided into an initial swing period T5, a mid-swing period T6, and a terminal swing period T7. FIG. 6 is an example, and does not limit the periods constituting one gait cycle, the names of these periods, and the like.

As illustrated in FIG. 6, in a gait, a plurality of events (also referred to as gait events) occur. P1 represents an event in which the heel of the right foot touches the ground (heel contact (HC)). The heel contact HC is also expressed as a heel strike (HS). P2 represents an event in which the toe of the left foot moves away from the ground while the sole of the right foot is in contact with the ground (opposite toe off (OTO)). P3 represents an event in which the heel of the right foot lifts with the sole of the right foot in contact with the ground (heel rise (HR)). P4 is an event in which the heel of the left foot is grounded (opposite heel strike (OHS)). P5 represents an event in which the toe of the right foot is separated from the ground in a state where the sole of the left foot is grounded (toe off (TO)). P6 represents an event in which the left foot and the right foot cross each other in a state where the sole of the left foot is grounded (foot adjacent (FA)). P7 represents an event in which the tibia of the right foot is approximately perpendicular to the ground with the sole of the left foot in contact with the ground (tibia vertical (TV)). P8 represents an event in which the heel of the right foot touches the ground (heel contact (HC)). P8 is relevant to the end point of the gait cycle starting from P1 and is relevant to the start point of the next gait cycle. FIG. 6 is an example, and does not limit events that occur during a gait or names of these events.

The transmission unit 13 (transmission means) outputs sensor data at a preset transmission timing. For example, the transmission unit 13 transmits sensor data via wireless communication. The sensor data transmitted from the transmission unit 13 is received by the gait index calculation device 15. The transmission timing of the sensor data is not limited. For example, the transmission unit 13 transmits the sensor data in real time in response to the measurement of the sensor data. For example, the transmission unit 13 may store sensor data measured during a predetermined period, and collectively transmit the stored sensor data at a preset timing. For example, the transmission unit 13 may transmit the sensor data in response to an instruction from the gait index calculation device 15.

For example, the transmission unit 13 transmits the sensor data to the gait index calculation device 15 via wireless communication. For example, the transmission unit 13 transmits the sensor data to the gait index calculation device 15 via a wireless communication function (not illustrated) conforming to a standard such as Bluetooth (registered trademark) or WiFi (registered trademark). The communication function of the transmission unit 13 may conform to a standard other than Bluetooth (registered trademark) or WiFi (registered trademark). The transmission unit 13 may transmit the sensor data to the gait index calculation device 15 via a wire such as a cable.

In the present example embodiment, the sensor 11 measures acceleration and angular velocity at a frequency lower than a normal measurement frequency. While a normal measurement frequency is about 100 Hz, the sensor 11 measures the acceleration and angular velocity at a low frequency of about 25 Hz. Therefore, according to the present example embodiment, the power consumption of the sensor 11 is suppressed. In addition, in the measurement device 10 of the gait measurement system 1 of the present example embodiment, a communication amount of sensor data is reduced. Therefore, according to the present example embodiment, power consumption in communication is also reduced. The sensor data transmitted from the transmission unit 13 is interpolated by the gait index calculation device 15.

### [Gait Index Calculation Device]

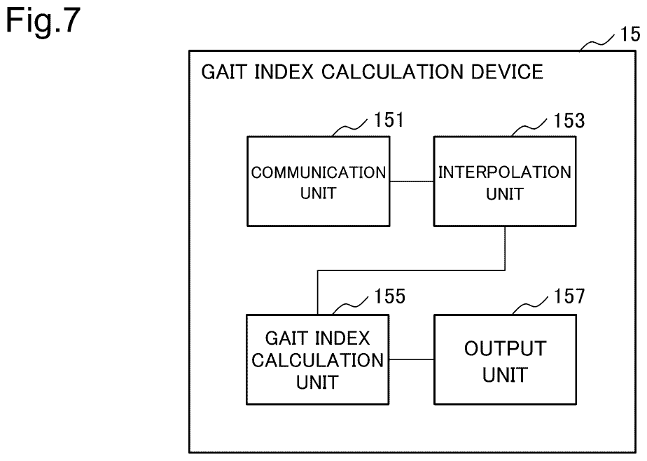

Next, a detailed configuration of the gait index calculation device 15 will be described with reference to the drawings. FIG. 7 is a block diagram illustrating an example of a configuration of the gait index calculation device 15. The gait index calculation device 15 includes a communication unit 151, an interpolation unit 153, a gait index calculation unit 155, and an output unit 157.

The communication unit 151 (communication means) receives the sensor data transmitted from the measurement device 10. For example, the communication unit 151 receives sensor data via wireless communication. The sensor data received by the communication unit 151 is subjected to data interpolation by the interpolation unit 153.

For example, the communication unit 151 receives the sensor data transmitted from the measurement device 10 via wireless communication. For example, the communication unit 151 receives sensor data from the measurement device 10 via a wireless communication function (not illustrated) conforming to a standard such as Bluetooth (registered trademark) or WiFi (registered trademark). The communication function of the communication unit 151 may conform to a standard other than Bluetooth (registered trademark) or WiFi (registered trademark). The communication unit 151 may receive the sensor data from the measurement device 10 via a wire such as a cable.

The communication unit 151 may have a function of transmitting a switching signal for switching the measurement frequency of the sensor data and a request signal for requesting transmission of the sensor data to the measurement device 10. For example, the communication unit 151 transmits a switching signal or a request signal in response to an instruction from the gait index calculation unit 155. For example, the communication unit 151 transmits a switching signal for switching the measurement frequency to a low frequency to the measurement device 10 in response to detection of a heel contact by the gait index calculation unit 155. For example, the communication unit 151 transmits a switching signal for switching the measurement frequency to a high frequency to the measurement device in response to the detection of the toe off by the gait index calculation unit 155.

The interpolation unit 153 (interpolation means) acquires sensor data. The interpolation unit 153 interpolates the received sensor data using a predetermined interpolation method. The interpolation unit 153 interpolates sensor data by inserting interpolation data between pieces of sensor data using a predetermined interpolation method. For example, the interpolation unit 153 interpolates the sensor data using an interpolation method such as linear interpolation, polynomial interpolation, or machine learning interpolation. For example, it is assumed that the measurement frequency of the sensor data is 25 Hz and the measurement frequency necessary for calculating the gait index is 100 Hz. In this case, the interpolation unit 153 inserts three pieces of interpolation data between the pieces of sensor data to obtain data equivalent to the data in the case where the measurement frequency is 100 Hz.

The sensor data transmitted from the measurement device 10 is based on acceleration and angular velocity measured at a frequency lower than a normal measurement frequency. Therefore, if the sensor data is used as it is, it is difficult to accurately calculate the gait index. For example, it is assumed that the normal measurement frequency is 100 Hz and the measurement frequency of the measurement device 10 is 25 Hz. In this case, if three pieces of data are interpolated between the pieces of sensor data generated by the measurement device 10, the gait index can be calculated with accuracy equivalent to that in the case where the measurement frequency is 100 Hz.

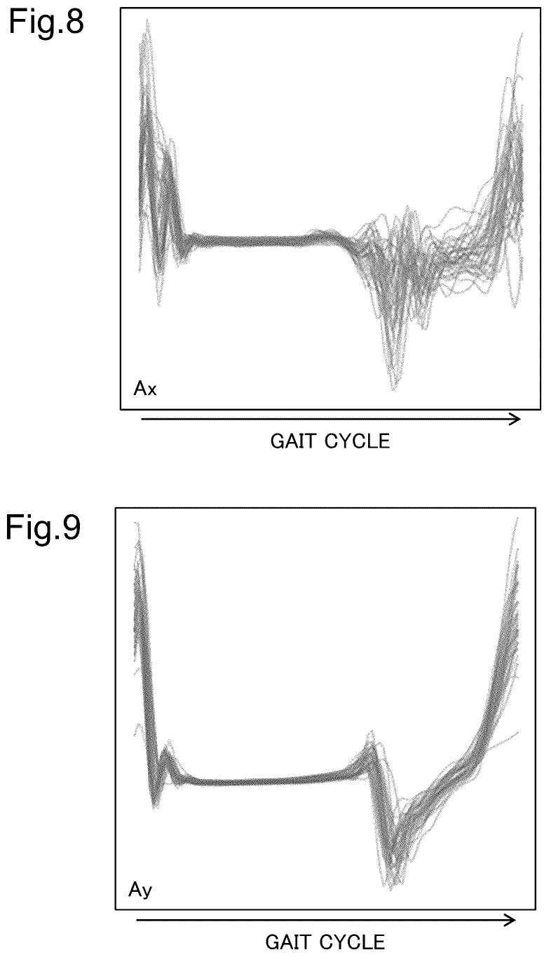

FIGS. 8 to 13 are graphs for explaining patterns of time-series data (also referred to as gait waveforms) of sensor data regarding acceleration and angular velocity measured for a plurality of subjects. FIGS. 8 to 13 are gait waveforms in one gait cycle with the timing of the heel contact as a start point and the timing of the next heel contact as an end point. The graphs in FIGS. 8 to 13 are normalized to the gait cycle with the timing of the heel contact as the start point (0%) and the timing of the next heel contact as the end point (100%). FIGS. 8 to 13 are graphs in which data of 720 points obtained by reciprocating three times of four trials of gait at a predetermined distance are superimposed for 60 subjects. The gait patterns for several gait cycles obtained in each trial are averaged in one gait cycle.

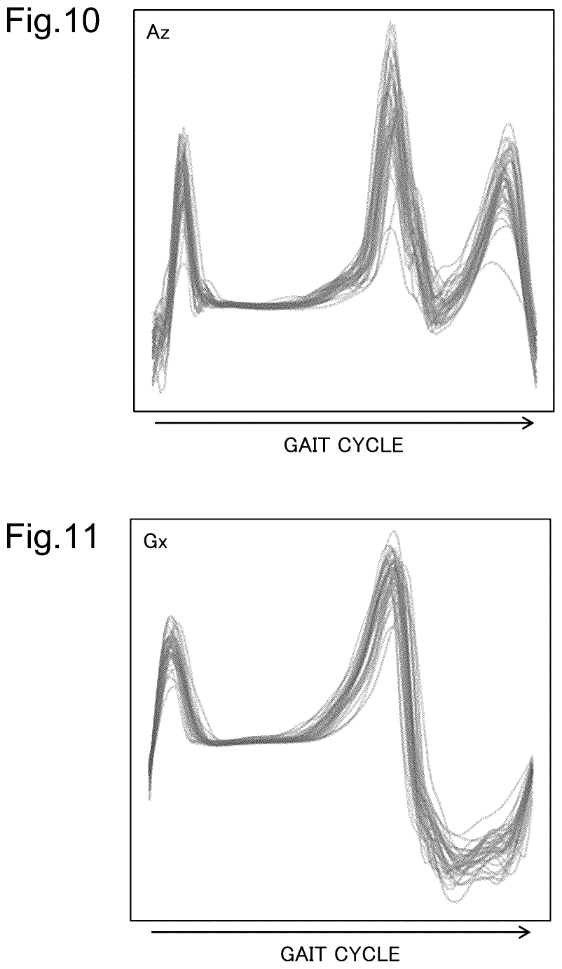

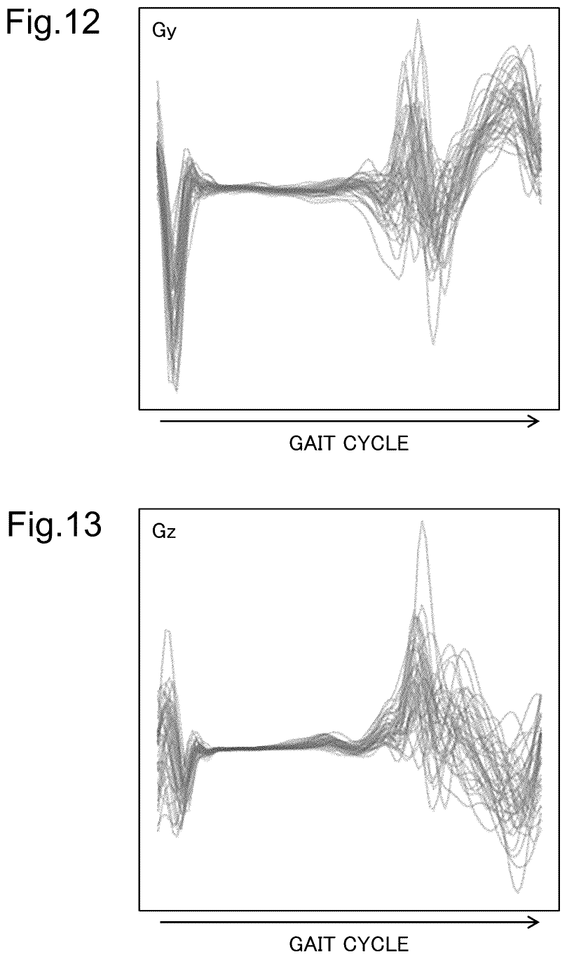

FIG. 8 is a gait waveform related to acceleration in the left-right direction x (left-right direction acceleration Ax). In the left-right direction x, the left is positive. FIG. 9 is a gait waveform related to acceleration in the traveling direction y (traveling direction acceleration Ay). In the traveling direction y, the backward direction is positive. FIG. 10 is a gait waveform related to acceleration in the vertical direction z (vertical direction acceleration Az). In the vertical direction z, the upward direction is positive. FIG. 11 is a gait waveform related to the angular velocity Gx about the left-right direction axis (about the x-axis). The angular velocity Gx is positive in a direction (clockwise) turning from the positive side of the y axis to the positive side of the z axis. FIG. 12 is a gait waveform related to the angular velocity Gy about the traveling direction axis (about the y axis). The angular velocity Gy is positive in a direction (clockwise) turning from the positive z-axis to the positive x-axis. FIG. 13 is a gait waveform related to the angular velocity Gz about the vertical direction axis (about the z axis). The angular velocity Gz is positive in a direction (clockwise) turning from the positive x-axis to the positive y-axis.

The gait waveform varies between on different subjects or even in the same subject. As illustrated in FIGS. 8 to 13, in any gait waveform, changes in acceleration and angular velocity are small in a state where the foot is in contact on the ground. In a section immediately after the heel contact (0 to 12%) and a period in which the foot is separated from the ground (50 to 100%), changes in acceleration and angular velocity are large. In a gait cycle in which these changes are large, a feature appears in the gait waveform. In particular, in the section (60 to 100%) of the swing phase, changes in acceleration and angular velocity are observed. In the section (60 to 100%) of the swing phase, the personality of the subject is seen.

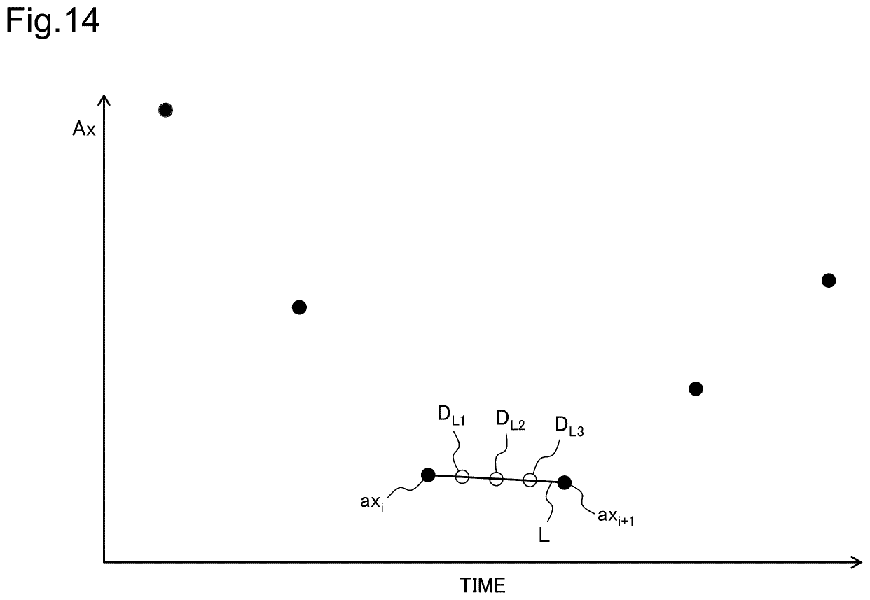

FIG. 14 is a graph for explaining linear interpolation related to the left-right direction acceleration Ax. In FIG. 14, sensor data based on actually measured acceleration is indicated by black circles, and interpolation data is indicated by white circles. FIG. 14 illustrates an example in which data is linearly interpolated between the acceleration axᵢ and the acceleration axᵢ₊₁ measured at consecutive timings (i is a natural number). In linear interpolation, the acceleration axᵢ and the acceleration axᵢ₊₁ are connected by a line segment L and equally divided at predetermined intervals. In the example of FIG. 14, the line segment L is divided into four equal parts by three pieces of interpolation data (D_{L1}, D_{L2}, D_{L3}). The linear interpolation is effective in a section in which a change is small or a section in which a change is monotonous. The linear interpolation can also be used for acceleration and angular velocity other than the left-right direction acceleration Ax similarly to the left-right direction acceleration Ax.

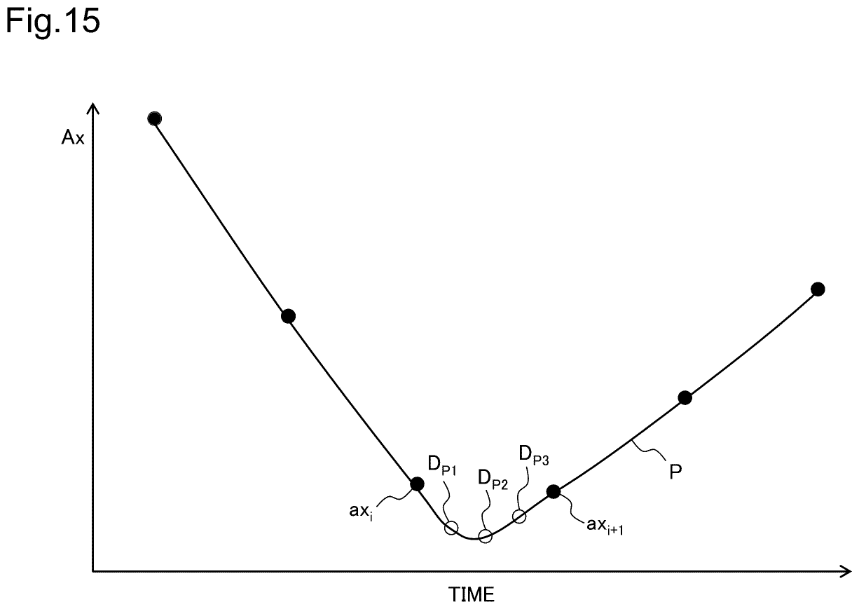

FIG. 15 is a graph for explaining polynomial interpolation related to the left-right direction acceleration Ax. In FIG. 15, sensor data based on actually measured acceleration is indicated by black circles, and interpolation data is indicated by white circles. FIG. 15 illustrates an example of polynomial interpolation of data between the acceleration axᵢ and the acceleration axᵢ₊₁ measured at consecutive timings (i is a natural number). In the polynomial interpolation, a polynomial curve P is fitted to acceleration measured at a plurality of close timings. The polynomial curve P in the section between the acceleration ax; and the acceleration axᵢ₊₁ is equally divided at predetermined intervals. In the example of FIG. 15, a section between the acceleration axᵢ and the acceleration axᵢ₊₁ is divided into four equal parts by three pieces of interpolation data (D_{P1}, D_{P2}, D_{P3}). The polynomial interpolation is valid in a section where the change is smooth. For example, the polynomial curve P is a curve expressed by a quadratic function. For example, the polynomial curve P may be a quadratic curve such as an ellipse or a hyperbola. For example, the polynomial curve P may be a curve represented by a cubic or higher function. As for the acceleration and the angular velocity other than the left-right direction acceleration Ax, polynomial interpolation can be used similarly to the left-right direction acceleration Ax.

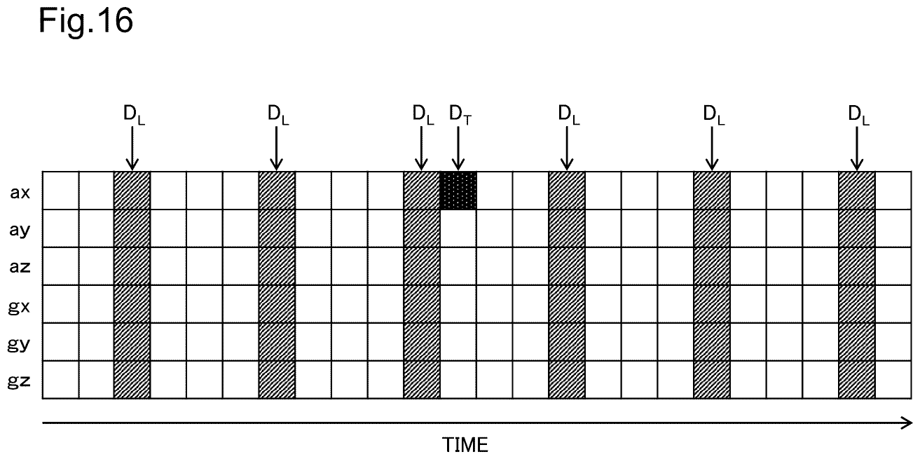

FIG. 16 is a conceptual diagram for describing machine learning interpolation. FIG. 16 illustrates a state in which sensor data (feature amounts) including the left-right direction acceleration ax, the traveling direction acceleration ay, the vertical direction acceleration az, the angular velocity gx about the left-right direction axis, the angular velocity gy about the traveling direction axis, and the angular velocity gz about the vertical direction axis are arranged at the same measurement timing. One square in the time axis direction indicates sensor data (feature amount) measured at a measurement frequency of 100 Hz. The learning data DI indicates sensor data (feature amount) measured at a measurement frequency of 25 Hz. The measurement interval of the measurement frequency of 100 Hz is 10 milliseconds. The measurement interval of the measurement frequency of 25 Hz is 40 milliseconds. In the machine learning interpolation, a model that outputs data between pieces of sensor data according to inputs of a plurality pieces of temporally continuous sensor data measured by the measurement device 10 is used. For example, the model outputs three pieces of interpolation data between pieces of sensor data in response to inputs of the sensor data measured at a measurement frequency of 25 Hz. As a result, sensor data related to a measurement frequency of 100 Hz is obtained using the measurement frequency of 25 Hz.

In the case of the example of FIG. 16, since each of the six pieces of learning data DL includes six feature amounts, a total of 36 feature amounts are used. The learning data DL is generated using a data set of sensor data measured at 100 Hz. Teacher data Dr is data of an interpolation region that is not measured at a measurement frequency of 25 Hz among data measured at a measurement frequency of 100 Hz. With respect to a cell of the teacher data DT, a model that outputs data (left-right direction acceleration ax) of the cell according to an input of 36 feature amounts is generated by learning using sensor data measured at 100 Hz. The interpolation region is a white square in FIG. 16 (including the square of the teacher data DT). In FIG. 16, only one point of the left-right direction acceleration ax is clearly indicated as the teacher data DT, but the model is generated for all the interpolation regions. In other words, the model is prepared for all the squares in the interpolation region.

For example, in learning of the left-right direction acceleration ax, all of the triaxial acceleration and the angular velocity about the three axes are used. In particular, in learning of the left-right direction acceleration ax, the traveling direction acceleration ay and the vertical direction acceleration az are important. That is, in the learning of the acceleration in a certain direction, the influence of the acceleration in the other two directions is strong in addition to the acceleration in the certain direction. Regarding the angular velocity about a certain axis, in addition to the angular velocity about the axis, the influence of the vertical direction acceleration az and the angular velocity about another axis is strong.

For example, the measurement frequency may be set smaller in a section in which the fluctuation of the acceleration or the angular velocity is small. Such a section may be switched from 25 Hz to 12.5 Hz, for example. The section in which the measurement frequency is reduced may be switched to linear interpolation instead of machine learning interpolation. Sufficient accuracy can be obtained even by linear interpolation in a section in which fluctuations of acceleration and angular velocity are small. When the machine learning interpolation and the linear interpolation are used together, the calculation load of the mobile terminal on which the gait index calculation device is mounted can be reduced.

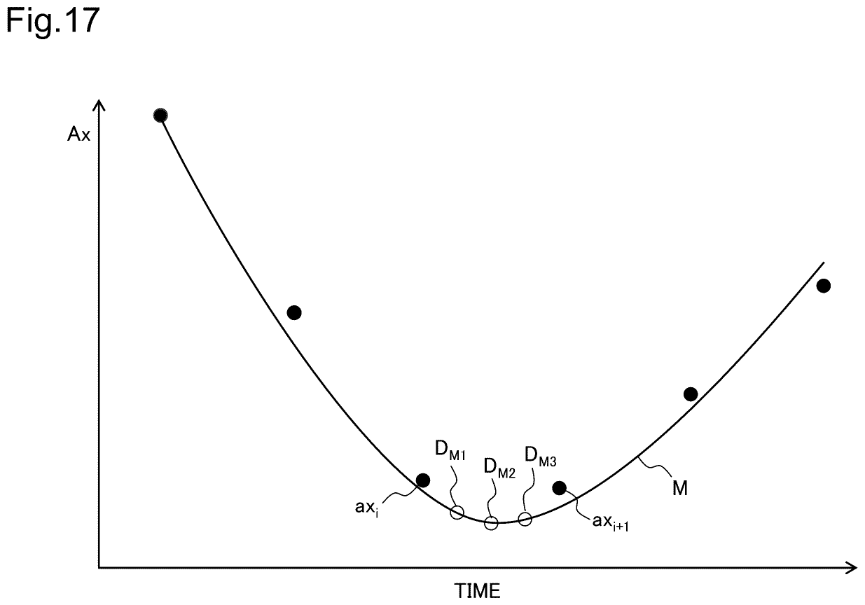

FIG. 17 is a graph for explaining machine learning interpolation related to the left-right direction acceleration Ax. In FIG. 17, sensor data based on actually measured acceleration is indicated by black circles, and interpolation data is indicated by white circles. FIG. 17 illustrates an example in which machine learning interpolation is performed on data between the acceleration axᵢ and the acceleration axᵢ₊₁ measured at consecutive timings (i is a natural number). In the machine learning interpolation, a curve M obtained by using machine learning is fitted to acceleration measured at a plurality of close timings. The curve M in the section between the acceleration axᵢ and the acceleration axᵢ₊₁ is equally divided at predetermined intervals. In the example of FIG. 17, a section between the acceleration axᵢ and the acceleration axᵢ₊₁ is divided into four equal parts by three pieces of interpolation data (D_{M1}, D_{M2}, D_{M3}). The machine learning interpolation is effective in both a section in which a change is smooth and a section in which a change is complicated. Regarding acceleration and angular velocity other than the left-right direction acceleration Ax, machine learning interpolation can be used similarly to the left-right direction acceleration Ax. The machine learning interpolation can interpolate sensor data more smoothly than linear interpolation or polynomial interpolation.

As illustrated in FIGS. 8 to 13, in a section (about 10 to 50%) in which the foot is in contact with the ground, variations in acceleration and angular velocity are small. In this section, even if the measurement frequency is reduced, the influence on the accuracy of the interpolation data is small. For example, in this section, the measurement frequency may be reduced from 25 Hz to 10 Hz or the like. When the measurement frequency is varied in this manner, a model is prepared for each measurement frequency. In other words, in the machine learning interpolation, a model is prepared for each measurement frequency.

In addition, whether the subject is walking may be determined according to the value of the sensor data. In this case, the measurement frequency may be changed according to the relationship between the preset threshold and the sensor data. For example, in a case where the value of the sensor data is less than the threshold value, an instruction may be issued from the gait index calculation device 15 to the measurement device 10 to drive in the low power consumption mode by lowering the measurement frequency.

The gait index calculation unit 155 (gait index calculation means) acquires sensor data in which data is interpolated by the interpolation unit 153. The gait index calculation unit 155 extracts time-series data (also referred to as gait waveform data) for one gait cycle from the time-series data of the acceleration in the three-axis direction and the angular velocity about the three axes included in the sensor data. The gait index calculation unit 155 normalizes (also referred to as first normalization) the time of the extracted gait waveform data for one gait cycle to a gait cycle of 0 to 100% (percent). Timing such as 1% or 10% included in the 0 to 100% gait cycle is also referred to as a gait phase. In addition, the gait index calculation unit 155 normalizes (also referred to as second normalization) the first normalized gait waveform data for one gait cycle in such a way that the stance phase becomes 60% and the swing phase becomes 40%. The stance phase is a period in which at least a part of the sole of the foot is in contact with the ground. The swing phase is a period in which the sole of the foot is off the ground. When the gait waveform data is subjected to the second normalization, it is possible to suppress the deviation of the gait phase from which the feature amount is extracted from fluctuating due to the influence of disturbance.

The gait index calculation unit 155 calculates a gait index by using the gait waveform data normalized for each gait cycle. As long as the gait waveform data normalized for each gait cycle can be calculated, the calculated gait index is not particularly limited. Hereinafter, representative gait indexes will be described. A specific calculation method of the following gait index will be omitted.

For example, the gait index calculation unit 155 calculates an index related to a distance and a height as a gait index. For example, the gait index calculation unit 155 calculates a stride length, an outward turning distance, a foot lifting height, a foot clearance (FTC), and a minimum toe clearance (MTC). The stride length indicates a distance between a front foot and a rear foot during walking. The outward turning distance indicates the maximum value of the distance at which the foot is separated outward with respect to the traveling direction in the swing phase. The foot lifting height indicates the maximum value of the distance between the measurement device 10 (sensor 11) and the ground in the swing phase. The FTC indicates the maximum distance between the heel and the ground in the swing phase. The MTC indicates the minimum value of the distance between the toe and the ground in the swing phase.

For example, the gait index calculation unit 155 calculates an index related to an angle as the gait index. For example, the gait index calculation unit 155 calculates the grounding angle, the ground separation angle, the direction of the toe, the roll angle of the heel contact, the roll angle of the toe off, the swing peak angular velocity, and the hallux angle. The grounding angle indicates a maximum value of an angle formed by the sole of the foot and the ground at the time of heel contact. The off ground angle indicates an angle formed by the sole of the foot and the ground in the swing phase. The direction of the toe indicates an average value of the directions of the toe with respect to the traveling direction in the swing phase. The roll angle of the heel contact is an angle formed between the ankle and the ground at the time of the heel contact when viewed from the rear viewing point. The roll angle of the toe off is an angle formed between the ankle and the ground at the time of kicking as viewed from the rear viewing point. The swing peak angular velocity is an angular velocity in the ankle joint dorsiflexion direction in a period from immediately after kicking until the toe comes into closest contact with the ground. The hallux angle indicates an angle at which the big toe of the foot is inclined toward the second toe. Specifically, the hallux angle is an angle formed by the center line of the first metatarsal and the center line of the first proximal phalange.

For example, the gait index calculation unit 155 calculates an index related to speed as the gait index. For example, the gait index calculation unit 155 calculates a gait speed, cadence, and a maximum speed while a swing. The gait speed indicates a speed in walking. The cadence indicates the number of steps per minute. The maximum speed while a swing indicates a speed at which the user swing phase.

For example, the gait index calculation unit 155 calculates an index related to time as the gait index. For example, the gait index calculation unit 155 calculates a stance time, a load applied time, a sole grounding time, a kicking time, a swing time, and a double support time (DST). The stance time indicates a time during which the foot is in contact with the ground during walking. The stance time is a sum of the load applied time, the sole grounding time, and the kicking time. The load applied time is a period from when the heel is in contact with the ground to when the toe is in contact with the ground in the stance phase. The sole grounding time is a time during which the entire sole surface is in contact with the ground and the sole surface and the ground are horizontal in the stance phase. The kicking time is a time until the toe kicks the ground from the state of the sole grounding in the stance phase. The swing time indicates a period during which the foot is off the ground during walking. The DST is divided into DST1 and DST2. The DST 1 indicates a time during which the foot on which the measurement device 10 (sensor 11) is mounted is in front of the opposite foot while both feet are simultaneously grounded to the ground. The DST 2 indicates a period during which the foot on which the measurement device 10 (sensor 11) is mounted is behind the opposite foot while both feet are simultaneously grounded to the ground.

For example, the gait index calculation unit 155 calculates a frailty level or a center of pressure exclusion index (CPEI) as the gait index. The frailty level is an estimated value of the frailty state according to the gait state. For example, the gait index calculation unit 155 estimates indexes such as a determination result A indicating health, a determination result B indicating a possibility of frailty, and a determination result C having a high possibility of frailty as the frailty level. The CPEI indicates an estimated value of the expansion ratio of the movement of the foot pressure center portion applied to the ground during the stance phase.

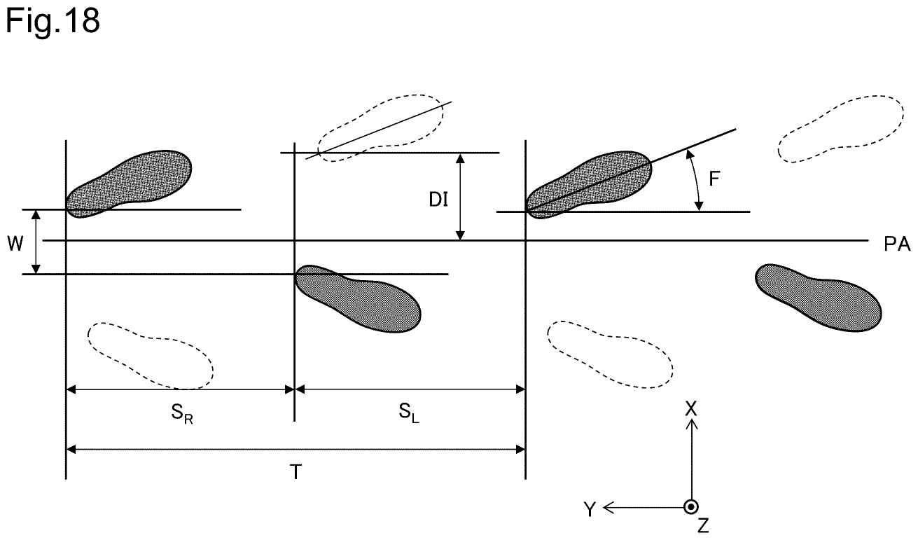

FIG. 18 is a conceptual diagram for describing an example of a gait index. FIG. 18 illustrates a right foot step length S_{R}, a left foot step length S_{L}, a stride length T, a step width W, a foot angle F, and a circumduction amount DI. In addition, FIG. 18 illustrates a traveling direction axis PA which is parallel to the axis (Y axis) in the traveling direction and is relevant to a trajectory connecting midpoints between the left foot and right foot. The right foot step length S_{R} is a difference in the Y coordinate between the heel of the right foot and the heel of the left foot in a transition from a state where the sole of the right foot is in contact with the ground to a state where the heel of the right foot swung out in the traveling direction is grounded. The left foot step length S_{L} is a difference in the Y coordinate between the heel of the left foot and the heel of the right foot in a transition from a state where the sole of the right foot is in contact with the ground to a state where the heel of the left foot swung out in the traveling direction is grounded. The stride length T is the sum of the right foot step length S_{R} and the left foot step length S_{L}. The step width W is an interval between the right foot and the left foot. In FIG. 18, the step width W is a difference between the center line (X-coordinate) of the heel of the right foot in the grounded state and the center line (X-coordinate) of the heel of the left foot in the grounded state. The foot angle F is an angle formed by the center line of the foot and the traveling direction (Y axis) in a state where the back of the foot is in contact with the ground. In the present example embodiment, the foot angle in a state where the foot is in contact with the ground is evaluated in the stance phase. The circumduction amount DI is a distance between the traveling direction axis PA and the foot at a timing when the central axis of the foot is farthest from the traveling direction axis PA in the swing phase. In the present example embodiment, since the length of the lower limb affects the circumduction amount DI, the circumduction amount DI is normalized by the height of their body.

For example, the gait index calculation unit 155 detects the timing of the heel contact HC and the toe off TO from the time-series data (solid line) of the traveling direction acceleration (acceleration in the Y direction). The timing of the heel contact HC is the timing of the local minimum peak immediately after the local maximum peak appearing in the time-series data of the traveling direction acceleration (acceleration in the Y direction). The local maximum peak serving as a mark of the timing of the heel contact HC is relevant to the maximum peak of the gait waveform data for one gait cycle. A section between the consecutive heel contacts HC is one gait cycle. The timing of the toe off TO is the rising timing of the local maximum peak appearing after the period of the stance phase in which the fluctuation does not appear in the time-series data of the traveling direction acceleration (acceleration in the Y direction).

For example, the gait index calculation unit 155 detects the timing of the mid-stance period from the time-series data (broken line) of the roll angle (angular velocity about the X axis). The timing at the midpoint between the timing at which the roll angle is minimum and the timing at which the roll angle is maximum is relevant to the mid-stance period. For example, parameters (also referred to as gait index) such as gait speed, stride length, circumduction, internal/external rotation, and plantarflexion/dorsiflexion can be obtained with reference to the mid-stance period.

For example, the gait index calculation unit 155 detects the heel contact HC and the toe off TO from the time-series data of the traveling direction acceleration (acceleration in the Y direction). The gait index calculation unit 155 extracts a section between consecutive heel contact HC as gait waveform data for one gait cycle. The gait index calculation unit 155 converts the left-right direction axis (time axis) of the gait waveform data for one gait cycle into a gait cycle of 0 to 100% by the first normalization. The gait index calculation unit 155 normalizes a section from the heel contact HC in which the gait phase is 0% to the toe off TO subsequent to the heel contact HC as 0 to 60%. In addition, the gait index calculation unit 155 normalizes a section from the toe off TO to the heel contact HC in which the gait phase subsequent to the toe off TO is at 100% as 60 to 100%. As a result, the gait waveform data for one gait cycle is normalized as a section (stance phase) in which the gait cycle is 0 to 60% and a section (swing phase) in which the gait cycle is 60 to 100%. In the gait waveform data (solid line) after the second normalization, the timing of the toe off TO coincides with 60%.

With respect to acceleration/angular velocity other than the traveling direction acceleration (acceleration in the Y direction), the gait index calculation unit 155 extracts/normalizes the gait waveform data for the gait cycle in accordance with the gait cycle of the acceleration in the traveling direction (acceleration in the Y direction). In addition, the gait index calculation unit 155 may generate time-series data of angles about three axes by integrating time-series data of angular velocities about the three axes. In that case, the gait index calculation unit 155 also extracts/normalizes the gait waveform data for one gait cycle in accordance with the gait cycle of the traveling direction acceleration (acceleration in the Y direction) with respect to the angle about the three axes.

The gait index calculation unit 155 may extract/normalize gait waveform data for one gait cycle based on acceleration/angular velocity other than the traveling direction acceleration (acceleration in the Y direction). For example, the gait index calculation unit 155 may detect the heel contact HC and the toe off TO from the time-series data of the vertical direction acceleration (Z-direction acceleration). The timing of the heel contact HC is a timing of a steep local minimum peak appearing in the time-series data of the vertical direction acceleration (Z-direction acceleration). At the timing of the steep local minimum peak, the value of the vertical direction acceleration (Z-direction acceleration) becomes substantially zero. The local minimum peak serving as a mark of the timing of the heel contact HC is relevant to the minimum peak of the gait waveform data for one gait cycle. A section between the consecutive heel contacts HC is one gait cycle. The timing of the toe off TO is a timing of an inflection point in the middle where the time series data of the vertical direction acceleration (Z-direction acceleration) gradually increasing after the local maximum peak immediately after the heel contact HC and then a section with a small fluctuation. The gait index calculation unit 155 may extract/normalize gait waveform data for one gait cycle based on both the traveling direction acceleration (acceleration in the Y direction) and the vertical direction acceleration (acceleration in the Z direction). The gait index calculation unit 155 may extract/normalize gait waveform data for one gait cycle based on acceleration, angular velocity, angle, and the like other than the traveling direction acceleration (acceleration in the Y direction) and the vertical direction acceleration (acceleration in the Z direction).

For example, the gait index calculation unit 155 extracts a feature amount (also referred to as a first feature amount) used for estimation of the gait index from the normalized gait waveform data for one gait cycle. The gait index calculation unit 155 extracts a feature amount for each gait phase cluster from a gait phase cluster obtained by integrating temporally continuous gait phases based on a preset condition. The gait phase indicates a timing such as 1% or 10% included in a 0 to 100% gait cycle. The gait phase cluster includes at least one gait phase. The gait phase cluster may be composed of a single gait phase. The gait waveform data and the gait phase of the extraction source of the feature amount used for estimating the gait index will be omitted.

For example, the gait index calculation unit 155 generates the feature amount (second feature amount) of the gait phase cluster by applying the feature amount constitutive formula to the feature amount (first feature amount) extracted from each of the gait phases constituting the gait phase cluster. The feature amount constitutive formula is a preset calculation expression for generating the feature amount of the gait phase cluster. For example, the feature amount constitutive formula is a calculation expression related to four arithmetic operations. For example, the second feature amount calculated using the feature amount constitutive formula is an integral average value, an arithmetic average value, a slope, a variation, or the like of the first feature amount in each gait phase included in the gait phase cluster. For example, the gait index calculation unit 155 applies a calculation expression for calculating the inclination or variation of the first feature amount extracted from each of the gait phases constituting the gait phase cluster as the feature amount constitutive formula. For example, in a case where the gait phase cluster is configured by a single gait phase, the inclination or variation cannot be calculated, and thus, it is sufficient to use a feature amount constitutive formula for calculating an integral average value, an arithmetic average value, or the like.

The gait index calculation unit 155 may be made to output, to the communication unit 151, a switching signal for switching a measurement frequency of sensor data or an instruction signal for instructing transmission of a request signal for requesting transmission of sensor data. For example, the gait index calculation unit 155 outputs an instruction signal for transmitting a switching signal for switching the measurement frequency to a low frequency (for example, 10 Hz or 12.5 Hz) to the communication unit 151 in response to the detection of the heel contact. For example, the gait index calculation unit 155 outputs an instruction signal for transmitting a switching signal for switching the measurement frequency to a high frequency (for example, 25 Hz) to the communication unit 151 in response to the detection of the toe off. Actually, the detection of the gait event by the gait index calculation unit 155 is transmitted from the measurement of the sensor data by the measurement device 10. Therefore, the gait index calculation unit 155 predicts the timing at which the heel contact and the toe off are actually measured based on the sensor data for several steps. The gait index calculation unit 155 outputs an instruction signal for instructing transmission of the switching signal to the communication unit 151 in such a way that the measurement frequency is switched in accordance with the timing of the heel contact or the toe off.

The output unit 157 (output means) outputs the gait index calculated by the gait index calculation unit 155. For example, the output unit 157 outputs the generated gait index to a terminal device (not illustrated) that displays the gait index. For example, the output unit 157 outputs the generated gait index to a device or system (not illustrated) that executes estimation using the gait index.

### (Operation)

Next, an operation of the gait measurement system 1 will be described with reference to the drawings. Hereinafter, the measurement device 10 and the gait index calculation device 15 included in the gait measurement system 1 will be individually described.

### [Measurement Device]

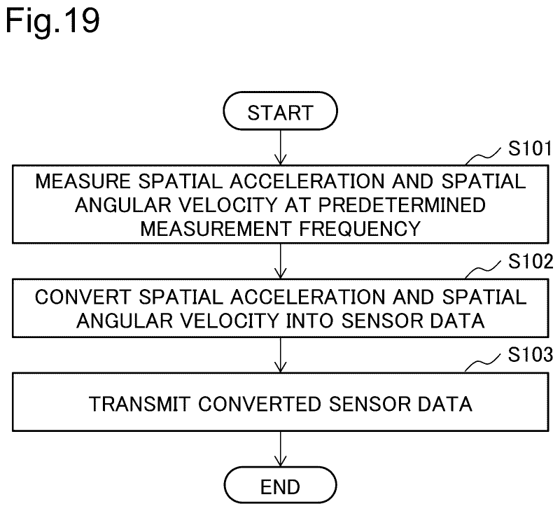

FIG. 19 is a flowchart for explaining an example of the operation of the measurement device 10. In the description along the flowchart of FIG. 19, the measurement device 10 will be described as an operation subject.

In FIG. 19, first, the measurement device 10 calculates a spatial acceleration and a spatial angular velocity at a preset measurement frequency (step S101). The measurement device 10 performs measurement at a measurement frequency lower than a measurement frequency at which a sufficient number of data for calculating the gait index can be obtained.

Next, the measurement device 10 converts the spatial acceleration and the spatial angular velocity into sensor data (step S102).

Next, the measurement device 10 transmits the converted sensor data to the gait index calculation device 15 (step S103).

### [Estimation Device]

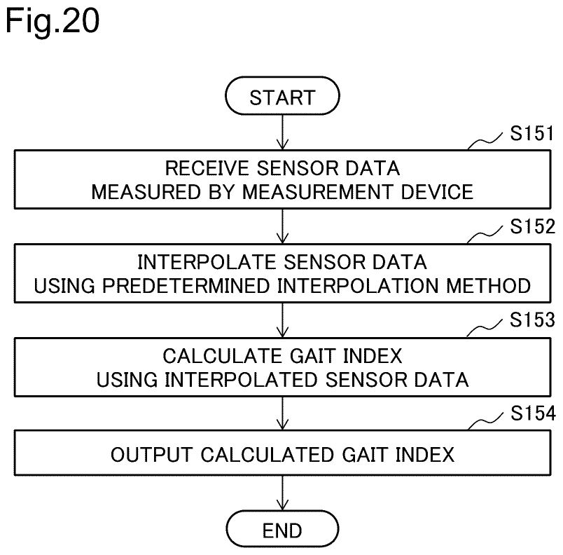

FIG. 20 is a flowchart for explaining an example of the operation of the gait index calculation device 15. In the description along the flowchart of FIG. 20, the gait index calculation device 15 will be described as an operation subject.

In FIG. 20, first, the gait index calculation device 15 receives sensor data measured by the measurement device 10 (step S151).

Next, the gait index calculation device 15 interpolates the sensor data using a predetermined interpolation method (step S152). For example, the gait index calculation device 15 interpolates the sensor data using a method of linear interpolation, polynomial interpolation, or machine learning interpolation.

Next, the gait index calculation device 15 calculates a gait index by using the interpolated sensor data (step S153). For example, the gait index calculation device 15 calculates a gait index regarding the distance, height, angle, speed, time, and the like.

Next, the gait index calculation device 15 outputs the calculated gait index (step S154). For example, the gait index calculation device 15 outputs the calculated gait index to a terminal device (not illustrated) that displays the gait index. For example, the gait index calculation device 15 outputs the calculated gait index to a device or system (not illustrated) that executes estimation using the gait index.

### Application Example

Next, an application example according to the present example embodiment will be described with reference to the drawings. In the following application example, an example in which the function of the gait index calculation device 15 installed in the mobile terminal carried by the user displays the calculation result of the gait index on the screen using the feature amount data measured by the measurement device 10 provided in the shoe will be described.

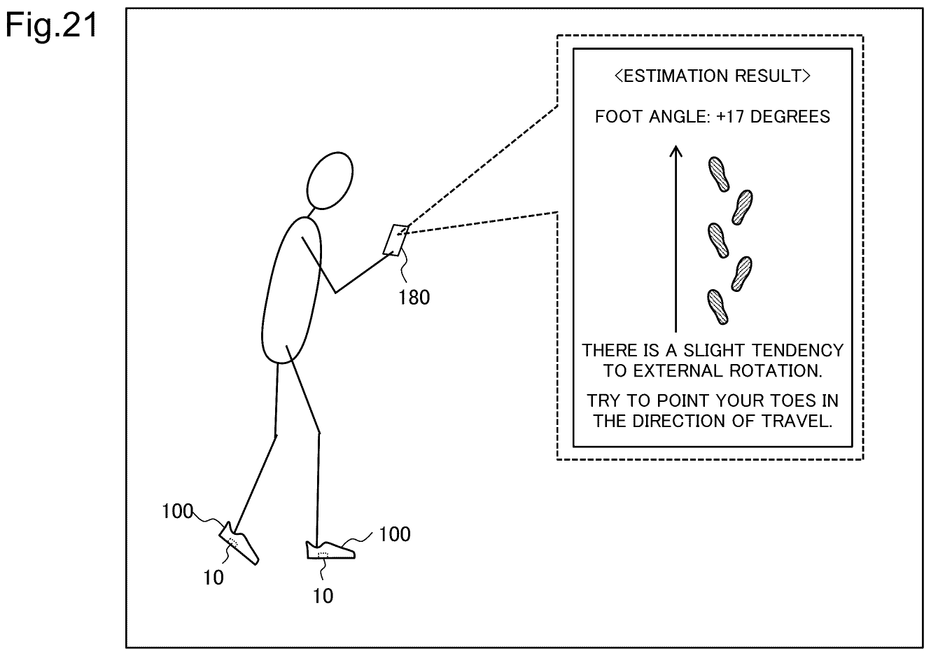

FIG. 21 is a conceptual diagram illustrating an example of displaying the calculation result by the gait index calculation device 15 on the screen of the mobile terminal 180 carried by the user walking while wearing the shoes 100 on which the measurement device 10 is provided. FIG. 21 is an example in which information related to a calculation result of a gait index according to sensor data measured according to gait of a user is displayed on a screen of the mobile terminal 180.

In the example of FIG. 21, the foot angle calculated by the gait index calculation device 15 and information related to the foot angle are displayed on the screen of the mobile terminal 180. FIG. 21 illustrates an example of displaying the foot angle (+17 degrees) calculated by the gait index calculation device 15 and the comment related to the foot angle (+17 degrees) on the screen. However, when the foot angle is +15 degrees or more, it is determined that “there is a slight tendency to external rotation”. For example, on the screen of the mobile terminal 180, information indicating whether the foot angle is prone to external rotation or internal rotation or information indicating advice on gait according to the foot angle is displayed as a comment according to the foot angle. In addition, in the example of FIG. 21, a foot type related to the foot angle of the walking person is displayed on the screen of the mobile terminal 180. In the example of FIG. 21, a foot type matching the foot angle of the walking person for each step is displayed on the screen.

As in the example of FIG. 21, the user who has visually recognized the information displayed on the screen of the mobile terminal 180 can estimate their gait state according to the information displayed on the screen. The information to be displayed on the screen is not limited to the example of FIG. 21 as long as the information related to the gait index.

As described above, the gait measurement system according to the present example embodiment includes the measurement device and the gait index calculation device. The measurement device includes a sensor and a transmission unit. The measurement device is installed on the footwear of the subject of the gait index measurement. The measurement device measures the spatial acceleration and the spatial angular velocity at a frequency lower than a normal measurement frequency. The measurement device outputs sensor data associated to their gait using the measured spatial acceleration and spatial angular velocity. The transmission unit transmits the sensor data output from the sensor to the gait index calculation device. The gait index calculation device includes the communication unit, the interpolation unit, the gait index calculation unit, and the output unit. The communication unit receives, from the measurement device, sensor data based on the spatial acceleration and the spatial angular velocity measured at a frequency lower than the normal measurement frequency. The interpolation unit interpolates the sensor data using a predetermined interpolation method. The gait index calculation unit calculates a gait index by using the interpolated sensor data. The output unit outputs the calculated gait index.

As described above, the measurement device included in the gait measurement device according to the present example embodiment measures the spatial acceleration and the spatial angular velocity at a frequency lower than the normal measurement frequency. Therefore, the power consumed by the measurement of the spatial acceleration and the spatial angular velocity is suppressed as compared with the case where the measurement is performed at the normal measurement frequency. In addition, since the communication load of the sensor data based on the spatial acceleration and the spatial angular velocity is reduced, the power consumption of the measurement device is suppressed as compared with the case of continuing measurement at a normal measurement frequency, and thus the measurement device can be used for a longer time. In addition, the gait measurement device included in the gait measurement device according to the present example embodiment receives sensor data measured by the measurement device. The communication load of the sensor data is reduced by receiving the sensor data based on the spatial acceleration and the spatial angular velocity measured at a frequency lower than the normal measurement frequency of the gait measurement device. In addition, the gait measurement device can calculate the gait index with the same accuracy as the normal measurement frequency by interpolating the sensor data using a predetermined interpolation method. That is, according to the present example embodiment, a communication load of sensor data can be reduced, and a gait index with sufficient accuracy can be calculated.

In one aspect of the present example embodiment, the interpolation unit interpolates the sensor data with at least one piece of data on a line segment connecting two pieces of temporally continuous sensor data. According to the present aspect, by interpolating interpolation data between sensor data by linear interpolation, a gait index can be calculated using sensor data of the same number of data as a normal measurement frequency. In particular, the method of the present aspect is effective in a section in which the fluctuation of the sensor data is small.

In one aspect of the present example embodiment, the interpolation unit interpolates the sensor data with at least one piece of data on a curve approximating a plurality of pieces of temporally continuous sensor data. According to the present aspect, by interpolating interpolation data between sensor data by polynomial interpolation, a gait index can be calculated using sensor data of the same number of data as a normal measurement frequency. In particular, the method of the present aspect is effective in a section where the fluctuation of the sensor data is large.

In one aspect of the present example embodiment, the interpolation unit interpolates sensor data using a model that outputs interpolation data of sensor data in accordance with inputs of a plurality of pieces of sensor data that are temporally close. According to the present aspect, by interpolating interpolation data between sensor data by machine learning interpolation, a gait index can be calculated using sensor data of the same number of data as a normal measurement frequency. In particular, the method of the present aspect is effective in a section in which it is difficult to predict sensor data from context and variation according to a specific pattern appears.

In one aspect of the present example embodiment, the communication unit transmits a switching signal for switching the measurement frequency to a measurement device that measures sensor data. The measurement device switches the measurement frequency in response to reception of the switching signal from the gait index calculation device. According to the present aspect, the power consumption of the measurement device can be suppressed by switching the measurement frequency according to the magnitude of the fluctuation of the sensor data.

In one aspect of the present example embodiment, the communication unit transmits a switching signal for switching the measurement frequency to a low frequency to the measurement device in response to detection of heel contact by the gait index calculation device. In addition, the communication unit transmits a switching signal for switching the measurement frequency to a high frequency to the measurement device in response to the detection of the toe separated place by the gait index calculation device. In the present aspect, the measurement frequency is switched to a low frequency in the section from the heel contact to the toe off where the fluctuation of the sensor data is small. In addition, in the present aspect, the measurement frequency is switched to the high frequency in the section from the toe off to the heel contact where the fluctuation of the sensor data is large. Therefore, according to the present aspect, it is possible to achieve both suppression of power consumption of the measurement device and calculation of the gait index with sufficient accuracy.

### Second Example Embodiment

Next, a gait index calculation device according to a second example embodiment will be described with reference to the drawings. The gait index calculation device according to the present example embodiment has a configuration in which the gait index calculation device included in the gait measurement system of the first example embodiment is simplified.

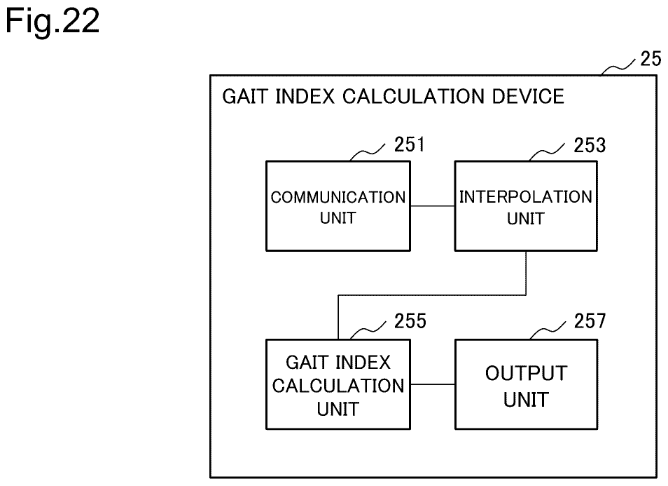

FIG. 22 is a block diagram illustrating an example of a configuration of a gait index calculation device 25 according to the present disclosure. The gait index calculation device 25 includes a communication unit 251, an interpolation unit 253, a gait index calculation unit 255, and an output unit 257.

The communication unit 251 receives sensor data based on spatial acceleration and spatial angular velocity measured at a frequency lower than a normal measurement frequency. The interpolation unit 253 interpolates the sensor data using a predetermined interpolation method. The gait index calculation unit 255 calculates a gait index by using the interpolated sensor data. The output unit 257 outputs the calculated gait index. As described above, in the present example embodiment, the sensor data communication load is reduced by receiving the sensor data based on the spatial acceleration and the spatial angular velocity measured at a frequency lower than the normal measurement frequency. In addition, in the present example embodiment, by interpolating the sensor data using a predetermined interpolation method, the gait index can be calculated with the same accuracy as that measured at the normal measurement frequency. That is, according to the present example embodiment, a communication load of sensor data can be reduced, and a gait index with sufficient accuracy can be calculated.

### (Hardware)

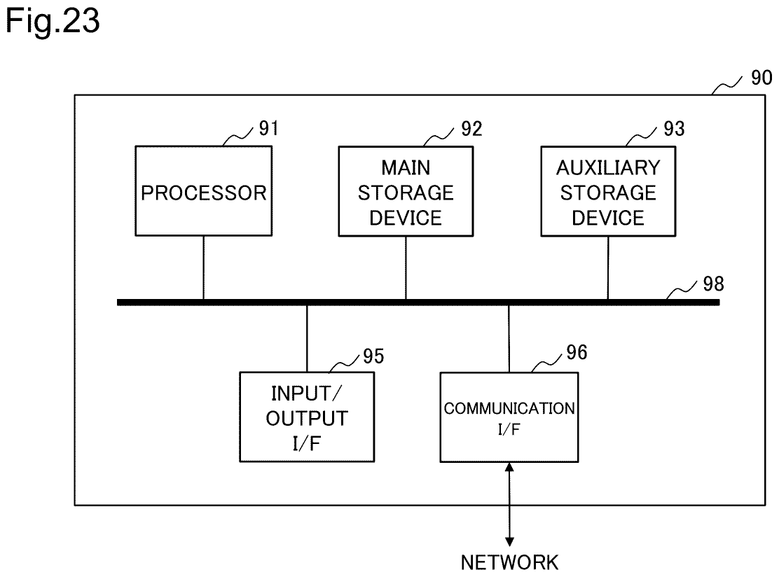

Next, a hardware configuration for executing control and processing according to each example embodiment of the present disclosure will be described with reference to the drawings. Here, an example of such a hardware configuration is an information processing device 90 (computer) in FIG. 23. The information processing device 90 in FIG. 23 is a configuration example for executing control and processing of each example embodiment, and does not limit the scope of the present disclosure.

As illustrated in FIG. 23, the information processing device 90 includes a processor 91, a main storage device 92, an auxiliary storage device 93, an input/output interface 95, and a communication interface 96. In FIG. 23, the interface is abbreviated as I/F. The processor 91, the main storage device 92, the auxiliary storage device 93, the input/output interface 95, and the communication interface 96 are data-communicably connected to each other via a bus 98. In addition, the processor 91, the main storage device 92, the auxiliary storage device 93, and the input/output interface 95 are connected to a network such as the Internet or an intranet via the communication interface 96.

The processor 91 develops a program (command) stored in the auxiliary storage device 93 or the like in the main storage device 92. For example, the program is a software program for executing control and processing of each example embodiment. The processor 91 executes the program developed in the main storage device 92. The processor 91 executes the program to execute control and processing according to each example embodiment.

The main storage device 92 has an area in which a program is developed. The program stored in the auxiliary storage device 93 or the like is developed in the main storage device 92 by the processor 91. The main storage device 92 is achieved by, for example, a volatile memory such as a dynamic random access memory (DRAM). In addition, a nonvolatile memory such as a magneto resistive random access memory (MRAM) may be configured or added as the main storage device 92.

The auxiliary storage device 93 stores various data such as programs. The auxiliary storage device 93 is achieved by a local disk such as a hard disk or a flash memory. Various data may be stored in the main storage device 92, and the auxiliary storage device 93 may be omitted.

The input/output interface 95 is an interface for connecting the information processing device 90 and a peripheral device based on a standard or a specification. The communication interface 96 is an interface for connecting to an external system or device through a network such as the Internet or an intranet based on a standard or a specification. The input/output interface 95 and the communication interface 96 may be shared as an interface connected to an external device.

Input devices such as a keyboard, a mouse, and a touch panel may be connected to the information processing device 90 as necessary. These input devices are used to input information and settings. When a touch panel is used as the input device, a screen having a touch panel function serves as the interface. The processor 91 and the input device are connected via the input/output interface 95.

The information processing device 90 may be provided with a display device for displaying information. In a case where a display device is provided, the information processing device 90 includes a display control device (not illustrated) for controlling display of the display device. The information processing device 90 and the display device are connected via the input/output interface 95.

The information processing device 90 may be provided with a drive device. The drive device mediates reading of data and a program stored in a recording medium and writing of a processing result of the information processing device 90 to the recording medium between the processor 91 and the recording medium (program recording medium). The information processing device 90 and the drive device are connected via an input/output interface 95.

The above is an example of the hardware configuration for enabling the control and processing according to each example embodiment of the present disclosure. The hardware configuration of FIG. 23 is an example of the hardware configuration for executing the control and processing according to each example embodiment, and does not limit the scope of the present disclosure. A program for causing a computer to execute the control and processing according to each example embodiment is also included in the scope of the present disclosure. A program recording medium in which the program according to each example embodiment is recorded is also included in the scope of the present disclosure. The recording medium can be achieved by, for example, an optical recording medium such as a compact disc (CD) or a digital versatile disc (DVD). The recording medium may be achieved by a semiconductor recording medium such as a universal serial bus (USB) memory or a secure digital (SD) card. The recording medium may be achieved by a magnetic recording medium such as a flexible disk, or another recording medium. In a case where the program executed by the processor is recorded in the recording medium, the recording medium is relevant to a program recording medium.

The components of each example embodiment may be combined. The components of each example embodiment may be implemented by software. The components of each example embodiment may be implemented by a circuit.

The previous description of embodiments is provided to enable a person skilled in the art to make and use the present invention. Moreover, various modifications to these example embodiments will be readily apparent to those skilled in the art, and the generic principles and specific examples defined herein may be applied to other embodiments without the use of inventive faculty. Therefore, the present invention is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. A gait index calculation device comprising:
a memory storing instructions, and
a processor connected to the memory and configured to execute the instructions to:
receive sensor data based on spatial acceleration and spatial angular velocity that are measured at a frequency lower than a normal measurement frequency;
interpolate the sensor data using a predetermined interpolation method;
calculate a gait index using the interpolated sensor data; and
output the calculated gait index.

2. The gait index calculation device according to claim 1, wherein
the processor is configured to execute the instructions to
interpolate the sensor data with at least one piece of data on a line segment connecting two pieces of the sensor data that are temporally continuous.

3. The gait index calculation device according to claim 1, wherein
the processor is configured to execute the instructions to
interpolate the sensor data with at least one piece of data on a curve approximating a plurality of pieces of the sensor data that are temporally continuous.

4. The gait index calculation device according to claim 1, wherein
the processor is configured to execute the instructions to
interpolate the sensor data using a model that outputs interpolation data for the sensor data according to inputs of a plurality of pieces of the sensor data that are temporally close.

5. The gait index calculation device according to claim 1, wherein
the processor is configured to execute the instructions to
transmit a switching signal for switching the measurement frequency to a measurement device that measures the sensor data.

6. The gait index calculation device according to claim 5, wherein
the processor is configured to execute the instructions to
transmit, to the measurement device, the switching signal for switching the measurement frequency to a lower frequency in response that the gait index calculation device detects a heel contact, and
transmit, to the measurement device, the switching signal for switching the measurement frequency to a higher frequency in response that the gait index calculation device detects a toe being off from the ground.

7. The gait index calculation device according to claim 1, wherein
the processor is configured to execute the instructions to
interpolate by machine learning, and
output advice information to help a subject of the gait index measurement to make decision according to the gait index.

8. A gait measurement system comprising:
the gait index calculation device according to claim 1; and
a measurement device that includes a sensor that is provided to footwear of a subject of the gait index measurement, measures spatial acceleration and spatial angular velocity at a frequency lower than a normal measurement frequency, and outputs sensor data regarding gait using the measured spatial acceleration and spatial angular velocity, and transmits the sensor data output from the sensor to the gait index calculation device.

9. The gait measurement system according to claim 8, wherein
the gait index calculation device transmits, to the measurement device, a switching signal for switching the measurement frequency, and
the measurement device switches the measurement frequency in response to the reception of the switching signal from the gait index calculation device.

10. A gait index calculation method that causes a computer to execute processing comprising:
receiving sensor data based on spatial acceleration and spatial angular velocity that are measured at a frequency lower than a normal measurement frequency;
interpolating the sensor data using a predetermined interpolation method;
calculating a gait index using the interpolated sensor data; and
outputting the calculated gait index.

11. A non-transitory recording medium in which a program is recorded, the program causing a computer to execute processing comprising:
receiving sensor data based on spatial acceleration and spatial angular velocity that are measured at a frequency lower than a normal measurement frequency;
interpolating the sensor data using a predetermined interpolation method;
calculating a gait index using the interpolated sensor data; and
outputting the calculated gait index.
